(19) United States
(12) Patent Application Publication
(10) Pub. No.: US 2009/0172843 A1
(43) Pub. Date: Jul. 2, 2009

(54) Specific Promoter Region Expresses in Actively Dividing Young Tissues and the Aging Tissues in Plants as Well as its Application

(75) Inventors: Wei-Fen Huang, Taipei County (TW); Yi-Yin Do, Taipei (TW); Pung-Ling Huang, Taipei (TW)

Correspondence Address: SCHMEISER OLSEN & WATTS, 18 E UNIVERSITY DRIVE, SUITE # 101, MESA, AZ, 85201, US

(73) Assignee: Agriculture and food Agency, Council of Agriculture, Excutive Yuan, Taipei (TW)

(21) Appl. No.: 12/134,056

(22) Filed: Jun. 5, 2008

(30) Foreign Application Priority Data
Dec. 27, 2007 (TW) 096150442

(51) Int. Cl. A01H 5/00; C12N 15/11; C12N 5/04; C12N 15/00

(52) U.S. Cl. 800/298; 536/24.1; 435/320.1; 435/419

(57) ABSTRACT

A promoter capable of activating expression specifically in both of the vigorously divisible young tissues and separate-related aging tissues in plants, characterized in that the promoter is a promoter of Oncidium ethylene receptor gene OgERS1, and has a sequence as shown in SEQ ID No: 3. A gene expression cassette is composed of a promoter having a DNA sequence as shown in SEQ ID No: 3, and a polynucleotide that is linked to the 3′ terminal of the promoter and has an open reading frame, wherein the promoter can activate the transcription of the polynucleotide in a organism having the gene expression cassette therein. A gene expression vector is composed of a promoter having a DNA sequence as shown in SEQ ID No: 3. A plant or parts of organ, tissue or cell of the plant has been transformed to contain the gene expression cassette described above.

### BACKGROUND OF THE INVENTION

1. Field of the Invention

The invention relates to a gene promoter with specificity, and in particular, to promoter-controlled gene expression in both of the actively dividing young tissues and separate-related aging tissues in plants as well as its application

2. Description of the Prior Art

When a genetic engineering technique is used for improving the character of an organism, or in carrying out relative research, a particular promoter sequence is usually used in promoting a gene to be expressed or studied. Accordingly, for a molecular biologist, more types of promoters each with different specificity indicate that there are more useful tools, as well as more conducive to the research and development of life science and biotechnology industry.

Heretofore, there are many examples using spatial-specific promoters (e.g., for tissues such as root, stem, leaf and the like), temporal-specific promoters (e.g., for various phases such as budding, blooming, fruiting and the like), or promoters inducible by specific substances (e.g., light of specific wavelength, low temperature, hormone and the like), for starting the expression of a transferred target gene, in order to regulate the gene expression and further to increase the economic efficiency of a crop (Masclaux et al., 2005; Moore et al., 2006).

Oncidium is one of the important cut flower exports in Taiwan. Because of the furcating characteristics in the inflorescence morphology of Oncidium, after harvesting, the pollinia cap of the flower tends to fall off due to pulling and dragging, which induces the biosynthesis of ethylene and hence accelerates aging (Huang, 1998; Lin, 1999). Accordingly, the inventor of this application carried out relative studies on the isolation and modulation of ethylene receptor gene (Huang, 2002) in order to select a promoter with tissue specificity by analyzing the promoter of the ethylene receptor gene.

In view of this, finding promoters with different specificity so as to start the expression of a transferred target gene at the target site is indeed one of the important topics for promoting the development of the biotechnology industry.

In view of the importance of developing promoters each with different specificity in the biotechnology industry, the inventor had sought to improve and innovate, and finally, after intensively studying for many years, successfully accomplished a promoter expressed specifically in both of the actively dividing young tissues and separate-related aging tissues in plants as well as its application according to the invention.

### SUMMARY OF THE INVENTION

The invention provides therefore a promoter with specificity. The promoter can activate expression in both of the actively dividing young tissues and separate-related aging tissues in plants.

The invention provides an application of the inventive specific promoter capable of activating expression in both of the actively dividing young tissues and separate-related aging tissues in plants. By means of the special tissue specificity of said promoter, a target gene can be expressed massively at these tissues of a plant.

The invention provides further a gene expression vector comprising the inventive specific promoter capable of activating expression in both of the actively dividing young tissues and separate-related aging tissues in plants. Said vector can transfer a target gene into a plant cell and further express massively target gene at said target plant tissues.

The specific promoter capable of activating expression in both of the actively dividing young tissues and separate-related aging tissues in plants is obtained from an ethylene receptor gene, OgERS1 (GeneBank accession number AF276233, SEQ ID No: 1), derived from a species of Oncidium (Oncidium “Gower Ramsey”). The specific promoter according to the invention is a 2,173 bp promoter sequence (SEQ ID No: 2) that is obtained by using a cDNA of Oncidium ethylene receptor gene as a probe, carrying out a plaque hybridization reaction with Oncidium genomic DNA library, and, after several purification, the resultant Oncidium ethylene receptor genomic clone being subjected to restriction enzyme map analysis and nucleic acid sequencing.

Then, polymerase chain reaction (PCR) is carried out using oligonucleotide primers that can be hybridized specifically under high stringency with ethylene receptor gene OgERS1 sequence to ligate said 2,173 bp promoter sequence (SEQ ID No: 2) with the 5′-end untranslated region (5′UTR) of Oncidium ethylene receptor gene OgERS1, namely, the 40 bp DNA before the translation starting site, ATG, within the exon 1 DNA and the exon 2 of the Oncidium ethylene receptor gene OgERS1. Subsequent to the ligation, an Oncidium ethylene receptor gene OgERS1 promoter (SEQ ID No: 3) is thus formed. In a preferred embodiment, said oligonucleotide primer possesses nucleotide sequences as shown in SEQ ID NO: 5, 6 and SEQ ID NO: 7 and 8.

In order to identify whether said Oncidium ethylene receptor gene OgERS1 promoter (SEQ ID No: 3) has tissue specificity, said promoter sequence is ligated to the 5′ terminal of a reporter gene β-glucuronidase (GUS) sequence (SEQ ID No: 4) to be used as a promoter for said reporter gene, and is constructed together into a commercial Agrobacterium tumefaciens transformed vector pBI101 (ClonTech) to give a plasmid pOgERS1-GUS. Then, by using Agrobacterium tumefaciens transformation, said plasmid pOgERS1-GUS is transformed into a model plant Arabidopsis thialana, and the promoter activity of said gene promoter is assayed by means of histochemical staining of GUS. The result indicates that said Oncidium ethylene receptor gene OgERS1 promoter (SEQ ID No: 3) can express the gene it promoted in both the actively dividing young tissues and separate-related aging tissues in plants. Accordingly, the inventive Oncidium ethylene receptor gene OgERS1 promoter (SEQ ID No: 3) is a promoter with extreme tissue specificity.

In addition to provide a specific promoter capable of activating expression in both the actively dividing young tissues and separate-related aging tissues in plants, the invention provides further a gene expression cassette comprising: (1) the inventive promoter sequence (SEQ ID No: 3), and (2) a polynucleotide with an open reading frame (ORF), i.e., a target gene, wherein said polynucleotide is linked to the 3′ terminal of the inventive promoter. Said promoter can activate transcription of said polynucleotide in an organism containing said gene expression cassette. In a preferred embodiment, said target gene is the reporter gene β-glucuronidase (GUS).

Additionally, by constructing the inventive Oncidium ethylene receptor gene OgERS1 promoter (SEQ ID No: 3) into a commercial Agrobacterium tumefaciens transformed vector, for example, pBI101 (ClonTech), pGREEN (GenBank Accession No: AJ007829), pGREEN II (GenBank Accession No: EF590266) (www.pGreen.ac.uk) and the like, a gene expression vector can be formed. Further, said target gene can be inserted into said gene expression vector such that, after ligating said target gene to the 3′ terminal of the inventive promoter, a gene expression cassette described above can be formed. Moreover, through Agrobacterium tumefaciens transformation, the inventive promoter together with the target gene linked to its 3′ terminal can be transformed into the target plant body and hence may alter the genomic constitution of the plant thus transformed. Therefore, the inventive promoter can activate effectively the expression of said target gene in said target transformed plant and the progeny thereof.

These features and advantages of the present invention will be fully understood and appreciated from the following detailed description of the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a restriction enzyme gene map of the inventive Oncidium ethylene receptor OgERS1 genomic clone λGOER20.

FIG. 1B is the construction strategy diagram of the plasmid pOgERS1-GUS for the inventive Oncidium ethylene receptor gene OgERS1 promoter.

FIG. 2 is the inventive Oncidium ethylene receptor gene OgERS1 promoter sequence (SEQ ID No: 3); where lower-cases indicates a 2,173 bp DNA sequence preceding the transcription start site of Oncidium ethylene receptor gene OgERS1. Upper-cases in a frame label the 110 bp DNA sequence of the exon 1 in the Oncidium ethylene receptor gene OgERS1. Upper-case underlined labels 40 bp DNA sequence preceding translation start site ATG of the exon 2 in Oncidium ethylene receptor gene OgERS1.

FIG. 3 shows the expression analysis of reporter gene β-glucuronidase (GUS) for the homozygote transformed progeny of Arabidopsis thialana (Columbia) OgERS1p::GUS at different growth and development stages. FIG. 3A: 10 days after sowing; FIG. 3B: 15 days after sowing; FIG. 3C: 20 days after sowing; FIG. 3D: 30 days after sowing; FIG. 3E: 45 days after sowing; wherein FIGS. 3A, 3B and 3C belong to vegetative growth stage; FIG. 3D begins to enter reproductive stage; FIG. 3E is in reproductive stage. GUS concentrates its activity at and near the meristem region where the cell division takes place the most vigorously, and shifts progressively to buds at the top end and various offshoots as the inflorescence developed.

FIG. 4 shows the expression analysis of reporter gene β-glucuronidase (GUS) at different tissues of the homozygote transformed progeny of Arabidopsis thialana (Columbia) OgERS1p::GUS 45 days after sowing. FIG. 4A: buds and auxiliary bud; FIG. 4B: receptacle and pedicel; FIG. 4C: abscission zone of the aging tissue of receptacle; FIG. 4D: abscission zone of silique.

### DETAILED DESCRIPTION OF THE PREFERRED EMBODIMENT

### EXAMPLE 1

### Cloning of Oncidium Ethylene Receptor Gene Promoter

### 1. The Source of OncidiumλEMBL3 Genomic Library

The Oncidium genomic library was prepared by extracting genomic DNA from leaves of Oncidium “Gower Ramsey”, and, by using bacteriophage λEMBL3 as the vehicle, replacing DNA fragment in enzymatic cleavage to construct genomic library.

### 2. Preparation and Labeling of Nucleic Acid Probe

Using cDNA (with sequence as shown in SEQ ID No: 1) of Oncidium ethylene receptor gene OgERS1 (GeneBank accession number AF276233) as the template, a random primer labeling method was employed to prepare a nucleic acid probe by means of Prime-A-Gene kit (Promega, USA) as follows: total reaction volume: 50 μL, comprising: 1×labeling buffer, pH6.6 {50 Mm Tris-HCl, pH 8.3, 5 mM MgCl₂, 2 mM DTT, 0.2M HEPES [N-(2-hydroxyethyl)piperazine-N′-(2-ethanesulfonic acid)], 26A₂₆₀ unit/mL of random hexadeoxyribonicleotides}, 20 μM each of dATP, dGTP, and dTTP, 500 ng/mL of denatured DNA template, 400 μg/mL of Bovine serum albumin (BSA), 50 μCi [α-³²P] dCTP (333 nM), and 5 units of Klenow DNA Polymerase. After reacting at 37° C. for 2 hours, 2 μL of 0.5M EDTA (pH8.0) was added to terminate the reaction, and then, 8 μL of tracing dye (50% glycerol, 0.25% bromophenol blue) was added. The reaction mixture was passed through Sephadex-G50 chromatographic column, and eluted with TE buffer solution (pH7.6). Every 160˜180 μL was collected into a tube, and the radioactivity of each tube was determined by Liquid Scintillation Counter (Beckman 1801). Appropriate fraction with the highest activity was used as the probe.

### 3. Screening of Oncidium Ethylene Receptor Genomic Library

A plaque hybridization method was employed to screen Oncidium genomic library. At first, E. coli strain XL1-Blue MRA (P2) was used as the transformation host of λEMBL3, which was cultured using NZY medium (each liter containing 5g NaCl, 2g MgSO₄-7H₂O, and 5 g yeast extract). Total of 1.5×10⁶ PFU (plaque forming unit) was screened under high stringency.

The bacteriophage was transferred onto nitrocellulose membrane. The transferred membrane was treated with denature buffer (0.5M NaOH, 1.5M NaCl) for 2 minutes, then with neutralization buffer [0.5M Tris base, 1.5M NaCl, 0.035% HCl (v/v)] for 5 minutes, and finally, immersed in 2× SSPE (1× SSPE, 0.18M NaCl, 10 mM NaH₂PO₄, 1 mM EDTA pH7.4) for 30 seconds. Thereafter, bacteriophage DNA was fixed in a vacuum oven at 80° C. for 2 hours. Then, it was placed in a solution containing 2× SSPE and 0.1% SDS, and slightly shaken at room temperature for 1 hour. The nitrocellulose membrane was then shifted into a pre-hybridization solution containing 5× SSPE, 5× BFP (1× BFP containing 0.02% BSA, 0.02% Ficoll-400,000, 0.02% PVP-360,000), 0.1% SDS, 50% formamide and 500 μg/mL of salmon sperm DNA, and was pre-hybridized at 42° C. for 2 hours. Then, a radioactive-labeled cDNA pOER23 was used as a probe and was hybridized with said membrane under conditions of 5× SSPE, 1× BFP, 0.1% SDS, 50% formamide and 100 μg/mL of salmon sperm DNA, at 42° C. for 16˜18 hours. Thereafter, the nitrocellulose membrane was treated twice with a wash buffer I (5× SSPE, 0.1% SDS) at room temperature each for 15 minutes. Next, the nitrocellulose membrane was treated twice with wash buffer II (1× SSPE, 0.5% SDS) at 37° C. each for 15 minutes to remove non-specific probe. After exposing to X-ray film (Kodak) at −80° C., bacteriophage containing target gene DNA could be detected. The bacteriophage was isolated from the medium, stored in a SM buffer solution containing 0.03% chloroform. After being purified several times, Oncidium ethylene receptor OgERS1 genomic clone λGOER20 was obtained.

### EXAMPLE 2

### Restriction Enzyme Map Analysis of Oncidium Ethylene Receptor Genomic Clone λGOER20

1. The Extraction of Bacteriophage DNA from the Clone λGOER20

DNA extraction was carried out on bacteriophage containing Oncidium ethylene receptor OgERS1 genomic clone λGOER20 obtained by screening as described in example 1 as follows. The bacteriophage and host cells (2×10⁹/mL) at bacterial count ratio of 5:1, was added 1 mL SM buffer solution and 5 mL of 2.5 mM CaCl₂. After mixing, the resultant mixture was placed at room temperature for 15 minutes, and then at 37° C. for 45 minutes. Then, the mixture was poured into 100 mL of 2× NZY liquid medium (0.4% MgSO₄—7H₂O, 2% NaCl, 1% bacto-yeast extract, 2% NZ amine, 0.2% casaimino acid, 5 mM MgSO₄, 25 mM Tris-HCl pH7.5), and cultured while shaken at 37° C. 240 rpm for more than 8 hours. After adding 4.5 mL chloroform, the mixture was treated by shaking at 37° C. 240 rpm for 15 minutes, and was centrifuged at 4° C. 7,000 rpm for 20 minutes (Beckman J2-MC, JA 10 rotor). The supernatant was removed and was added with 100 μL DNase I (1 mg/mL) and 100 μL RNaseA (10 mg/mL). After treated at 37° C. 80 rpm for 45 minutes, 33 mL of 4 M NaCl was added and was incubated in an ice bath for 1 hour. Thereafter, 33 mL of ice-cold 50% polyethylene glycol was added and precipitated at 4° C. overnight. Then, the mixture was centrifuged at 4° C. 5,000 rpm for 20 minutes (Beckman J2-MC, JA 10 rotor), and the supernatant was discarded. After being air-dried, 500 μL of PKB solution (10 mM NaCl, 10 mM Tris-HCl pH 8.0, 10 mM EDTA, 0.1% SDS) was added to re-suspend the precipitate. Next, proteinase K (final concentration: 12.5 μg/mL) was added, and the mixture was allowed to react at 37° C. for 20 minutes. The reaction mixtures were extracted successively with equal volume of phenol, PCI (phenol: chloroform: isoamyl alcohol=25: 24: 1), and CI (chloroform: isoamyl alcohol=24: 1). The reaction mixture was centrifuged at room temperature at 14,000 rpm for 5 minutes. 2-fold volume of −20° C. 100% ethanol was added to the supernatant. After shaking homogeneously, DNA was picked out with a bended glass rod and was air-dried. The residue was centrifuged at 4° C. 14,000 rpm for 10 minutes. After decanting off the supernatant, the precipitate was air-dried. The combined two DNA precipitates were washed successively with 70% ethanol and 100% ethanol to remove the salt. The product thus obtained was dissolved in TE buffer solution (pH7.5), and was stored at 4° C. until used.

### 2. Restriction Enzyme Map Analysis

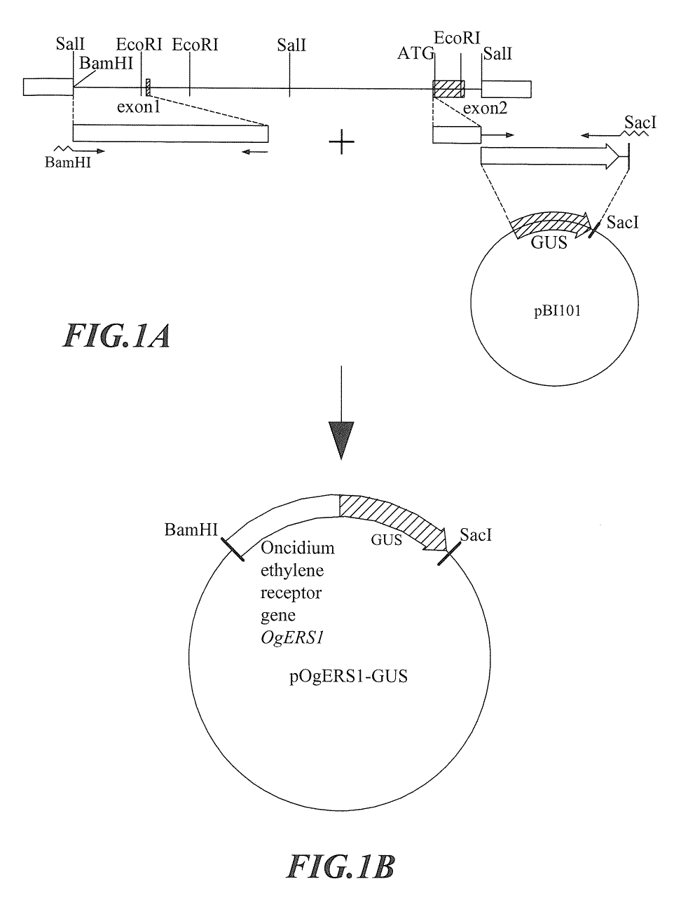

DNA of clone λGOER20 extracted as described above was subjected to cleavage with restriction enzymes SalI, BamHI, EcoRI, SalI/ BamHI, BamHI/EcoRI and SalI/EcoRI. After separating by 0.7% agarose gel electrophoresis, the resulting DNA fragment was transferred onto a nylon membrane Hybond-N (Amersham). After transferring, the nylon membrane was pre-hybridized in a pre-hybridization solution (containing 5× SSPE, 5× BFP, 0.5% SDS, 50% formamide, 250 μg/mL of salmon sperm DNA) at 42° C. for 2 hours. Thereafter, by using, separately, ³²P-labeled pOER23 cDNA 5′ terminal, including: (1) 825 bp DNA fragment recovered after EcoRI mono-cleavage; (2) 288 bp DNA fragment recovered after EcoRV/XhoI double cleavage; (3) 100 bp DNA fragment recovered after EcoRI/EcoRV double cleavage; as well as pOER23 cDNA 3′ terminal fragment, including: (1) 1605 bp DNA fragment after EcoRI/XhoI double cleavage; (2) 1154 bp DNA fragment recovered after AvaI/DraII double cleavage, as the probes, a hybridization reaction was carried out in a hybridization solution (containing 5× SSPE, 5× BFP, 0.5% SDS, 50% formamide, 100 μg/mL of salmon sperm DNA) at 42° C. for 16˜18 hours. As the reaction was terminated, the reaction mixture was treated twice with washing solution I (2× SSPE, 0.1% SDS) at room temperature each for 15 minutes, and then twice with washing solution II (1× SSPE, 0.1% SDS) at 65° C. each for 15 minutes to remove non-specific probe. After exposing to X-ray film (Kodak) at −80° C., and in conjunction with fluorescence electrophoresis photograph, various restriction maps could be plotted for each restriction fragment. The results were shown in FIG. 1A.

### 3. Sequencing of DNA

DNA was sequenced on an automated nucleic acid sequencer (ABI sequencer 377) to obtain the sequence of Oncidium ethylene receptor OgERS1 genomic clone λGOER20, and was analyzed with a PC/Gene software package from IntelliGenetics Inc. The results were shown in FIG. 1A. Oncidium ethylene receptor OgERS1 genomic clone λGOER20 had two exon, namely, exon 1 and exon 2, respectively, the translation starting site (codon encoding ATG) located at 42-44th nucleotides of exon 2, an intorn 1 of about 8.2 kb in length between the exon 1 and the exon 2; as well as the 2,173 bp promoter region was located in the upstream of the transcription start site on the exon 1 (i.e., the first nucleotide sequence on the exon 1) of Oncidium ethylene receptor OgERS1 genomic clone λGOER20. The sequence of said promoter region was shown as in SEQ ID No: 2.

### 4. Sequence Analysis of Oncidium OgERS1 Promoter

The promoter sequence thus obtained was input in PlantCARE databank, and search the characteristics of the promoter sequence was carried out (http://sphinx.rug.ac.be:8080/PlantCARE/index.htm). The result was shown in Table 1. From these, it was speculated that the −91˜−98 bp region from the initial point of Oncidium ethylene receptor gene cDNA was a TATA box, while the translation starting site was located at about 8.9 kb following said TATA box. In addition, the result of sequence analysis revealed that Oncidium ethylene receptor gene promoter possessed a number of response elements, wherein, other than ethylene-responsive element (ERE) involved in the regulation by ethylene, there were one AuxRR-core motif affected by auxin, 2 CGTCA-motifs associated with the response with jasmonate, 1 ABREs motif involved in the modulation by abscisic acid (ABA), 1 LTR-motif associated with low temperature, 1 ELI-box3 responsive to elicitor, 7 wound inducible factors WUN-motif, and a plurality of HSE-motifs responsive to high temperature adverse circumstance. Furthermore, there were several promoter conserved sequences associated with light response, such as ACE, AT1-motif, ATC-motif, CATT-motif, G-Box, GA-motif, GAG-motif, GT1-motif, Gap-box, I-box, LAMP-element, MRE, TCCC-motif, TCT-motif, TGG-motif, chs-CMA1a and the like. There were many environmental and physiological factors involved in the regulation of OgERS1 promoter activity.

TABLE 1
Sequence analysis of Oncidium ethylene receptor gene promoter.

| | Sequence in | Position in | |
|---|---|---|---|
| Motif | OgERS1 | OgERS1 | Regulatory function |
| ABRE | AACGTGT | −133~−139 | cis-acting element involved in |
| | | | the abscisic acid responsiveness |
| AuxRR-core | GGTCCAG | −2054~−2044 | cis-acting regulatory element |
| | | | involved in auxin |
| | | | responsiveness |
| CGTCA-motif | CGTCA | −1067~−1071 | cis-acting regulatory element |
| | CGTCA | −1979~−1975 | involved in the |
| | | | MeJA-responsiveness |
| ERE | ATTTCAAA | −760~−767 | ethylene-responsive element |
| | ATTTTAAA | −1337~−1344 | |
| | ATTTCAAC | −1454~−1461 | |
| LTR | CCGAAA | −1016~−1021 | cis-acting element involved in |
| | | | low-temperature responsiveness |
| WUN-motif | TAATTACAA | −591~−599 | wound-responsive element |
| | ATATTTCAA | −760~−768 | |
| | TAATTTCTT | −803~−811 | |
| | TCATTACAC | −1125~−1133 | |
| | AAATTTCTC | −1304~−1312 | |
| | AAATTGCCA | −1318~−1326 | |
| | TAATTACAT | −1356~−1364 | |
| | GCATTTCAA | −1455~−1463 | |
| | TCATTACCT | −2101~−2109 | |
| ELI-box3 | AAACTAATT | −807~−814 | elicitor-responsive element |
| HSE | TGAAAATTT | −474~−482 | cis-acting element involved in |
| | AGAAATTTA | −604~−612 | heat stress responsiveness |
| | AAAAAATGG | −893~−901 | |
| | AAAAAATAT | −1044~−1052 | |
| | AAAAAAGTTA | −1116~−1124 | |
| | TGAAAATTT | −1181~−1189 | |
| | AAAAAATGA | −1190~−1198 | |
| | ATAAAATTT | −1271~−1279 | |
| | AAAAAATAT | −1293~−1301 | |
| | TAAAAATTTT | −1331~−1340 | |
| | TAAAACTAT | −1368~−1376 | |
| | TGAAATTTT | −1387~−1396 | |

### EXAMPLE 3

### Construction of Vector Containing Oncidium Ethylene Receptor Gene OgERS1 Promoter

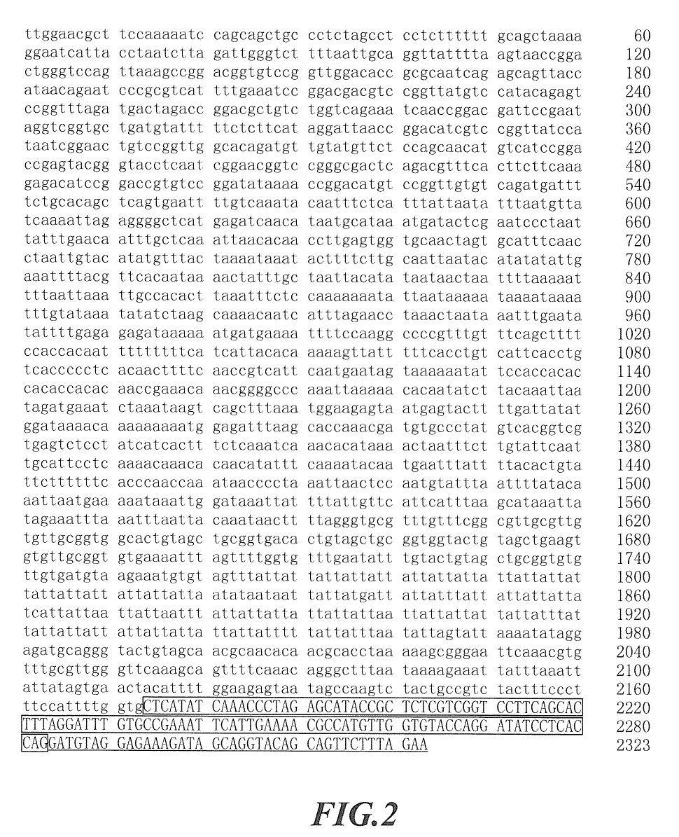

The construction strategy of Oncidium ethylene receptor gene OgERS1 promoter was shown as in FIG. 1B. The 2,173 bp sequence preceding the transcription start site of Oncidium ethylene receptor gene OgERS1, the total length 110 bp of the exon 1 DNA, and the 40 bp DNA sequence preceding the translation start site ATG in the exon 2 were ligated together to form Oncidium ethylene receptor gene OgERS1 promoter (its DNA sequence was shown in FIG. 2 and in SEQ ID No: 3), and then was constructed together into a commercial Agrobacterium tumefaciens transformed vector pBI101 (ClonTech). Said Oncidium ethylene receptor gene OgERS1 promoter (SEQ ID No: 3) was then ligated to the 5′ terminal of the sequence of a reporter gene λ-glucuronidase (GUS) (SEQ ID No: 4), to be used as the promoter of said reporter gene.

### Step 1: Obtaining the 2,173 bp Region Sequence Preceding the Transcription Start Site of Oncidium Ethylene Receptor Gene OgERS1 and the Exon 1 DNA Sequence

By using the genomic DNA extracted from leaves of Oncidium “Gower Ramsey” as the template, a polymerase chain reaction (PCR) was carried out to amplify the 2,173 bp region sequence preceding the transcription start site of Oncidium ethylene receptor gene OgERS1 and the exon 1 DNA sequence. Primer sequences used in the PCR were as followed:

forward primer (containing BamHI restriction enzyme cleavage site):

| | |
|---|---|
| 5′-TGC [symbol] TTGGAACGCTTCCAAAAATC-3′ | (SEQ ID No: 5) |
| BamHI | |
| reverse primer | |
| 5′-CCAGGATATCCTCACCAG-3′ | (SEQ ID No: 6) |

The total reaction volume of PCR was 50 μl (containing: 1 μl genomic DNA, 10 μ5×Phusion HF buffer, 1 μl of 10 mM dNTP, 1 μl of 20 μM forward primer, 1 μl of 20 tM reverse primer, 35.5 μl sterile water, 0.5 μl Phusion DNA polymerase). PCR reaction conditions were: 98° C. for 30 seconds, then 35 cycles of 98° C. 10 seconds, 69° C. 30 seconds, and 72° C. 60 seconds, and finally at 72° C. for 10 minutes for elongation. The PCR products were subjected to restrictive cleavage with BamHI restriction enzyme. A DNA fragment of 2,288 bp in length was recovered and stored at 4° C. until used.

### Step 2: Obtaining 40 bp DNA Sequence Preceding the Translation Start Site of the Exon 2 in Oncidium Ethylener Receptor Gene OgERS1

By using a commercial Agrobacterium tumefaciens transformed vector pBI101 (ClonTech) as the template, a PCR was carried out to amplify the 40 bp region sequence preceding the translation start site of the exon 2 in the Oncidium ethylene receptor gene OgERS1 and DNA sequence of a reporter gene μ-glucuronidase (GUS). Primer sequences used in PCR were as follows:

forward primer [containing the 40 bp preceding the translation start site of the exon 2 in Oncidium ethylene receptor gene OgERS1 (i.e., upper-case underlined sequence) and the sequence linked with the 5′ terminal of GUS gene (i.e., lower-case labeled sequence)]:

(SEQ ID No: 7)

5′-GATGTAGGAGAAAGATAGCAGGTACAGCAGTTCTTTAGAAatgttac
gtcctgtag-3′

reverse primer (complementary with the 3′ sequence of GUS gene, said sequence itself containing SacI restriction enzyme cleavage site):

| | |
|---|---|
| 5′-gcctcgggaattgctaccgagctcgaa-3′ | (SEQ ID No: 8) |
| SacI | |

The total volume of PCR was 50 μl (containing: 1 μl genomic DNA, 10 μl of 5× Phusion HF buffer, 1 μl of 10 mM dNTP, 1 μl of 20 μM forward primer, 1 μl of 20 μM reverse primer, 35.5 μl of sterile water, 0.5 μl Phusion DNA polymerase). The PCR reaction conditions were: 98° C. 30 seconds, then 35 cycles of 98° C. 10 seconds, 69° C. 30 seconds, and 72° C. 30 seconds, and finally 72° C. 10 minutes for elongation. The PCR product was subjected to enzymatic cleavage with SacI restriction enzyme. A DNA fragment of 1,908 bp in length was recovered and stored at 4° C. until used.

### Step 3: Ligation of DNA

A commercial Agrobacterium tumefaciens transformed vector pBI101 (ClonTech) was subjected to double enzymatic cleavage with BamHI+SacI restriction enzymes. The vector pBI101 was then recovered and was subjected to DNA ligation with DNA fragment of 2,288 bp in length prepared in Step 1 and the DNA fragment of 1,908 bp in length obtained in Step 2 to give a plasmid pOgERS1-GUS containing Oncidium ethylene receptor gene OgERS1 promoter sequence (as shown in SEQ ID No: 3). In said plasmid pOgERS1-GUS, the 3′ terminal of Oncidium ethylene receptor gene OgERS1 promoter was ligated further with a DNA sequence (SEQ ID No: 4) of a reporter gene λ-glucuronidase (GUS). Consequently, after transforming said plasmid pOgERS1-GUS into Agrobacterium tumefaciens through Arabidopsis inflorescence infiltration, the mode that Oncidium ethylene receptor gene OgERS1 promoter activated the gene expression of reporter gene λ-glucuronidase (GUS) could be analyzed.

### EXAMPLE 4

### Transformation of Arabidopsis Thialana (Columbia) by Applying Agrobacterium Tumefaciens—Mediated Transformation

By using a model plant, Arabidopsis thialana (Columbia) as the material, and employing Agrobacterium tumefaciens inflorescence infiltration method(Clough and Bent, 1998), the plasmid pOgERS1-GUS prepared in Example 3 was transformed into Arabidopsis thialana (Columbia), thereby changed the genomic constitution of the plant thus transformed such that Oncidium ethylene receptor gene OgERS1 promoter could activate effectively the expression of the reporter gene GUS in the transformed plant itself as well as in its progeny. Moreover, the performance that the reporter gene GUS was expressed in Arabidopsis thialana (Columbia) transformant was analyzed by histochemical staining of GUS so as to detect whether the Oncidium ethylene receptor gene OgERS1 promoter had tissue specificity.

### 1. The Growth Condition of Arabidopsis Thialana (Columbia)

The seeds were treated at 4° C. for 2˜4 days, and then sowed in a medium consisted of peat, Perlite, and vermiculite in a ratio of 10:1:1. Growth conditions were: temperature of 22˜25° C., light period of 16 hours, and 75% relative humidity. After 4-6 weeks, the plant was pruned. As the rachis had grown to a length of about 3 inches 4-8 days after pruning, the plant was subjected to transformation.

### 2. Preparation of Agrobacterium Tumefaciens Competent Cell and Transformation

Agrobacterium tumefaciens LBA4404 strain was inoculated in YEB solid medium (0.5% beef extract, 0.1% yeast extract, 0.5% peptone, 0.5% mannitol, 0.05% MgSO₄, 1.25% agar, pH 7.5) supplemented with suitable antibiotics (50 μg/ml of kanamycin, 50 μg/ml of ampicillin), and cultivated at 28° C. for 2 days. Then, a single colony was picked and inoculated in 20 ml of YEB liquid medium containing suitable antibiotics (50 μg/ml of kanamycin, 50 μg/ml of ampicillin). After incubation under shaking at 28° C. 240 rpm for 1 day, 5 ml of the culture suspension was added into 200 ml of YEB liquid medium and the resulting suspension was cultured by shaking at 28° C. 240 rpm for 9 hours. The suspension thus obtained was centrifuged at 4° C. 4,200 rpm for 20 minutes (Beckman J2-MC, JA-10 rotor). The supernatant was discarded and the pellet was suspended in 20 ml pre-chilled YEB medium. The suspension was centrifuged again at 4° C. 4,200 rpm for 20 minutes. The pellet was re-suspended in 20 ml pre-chilled YEB medium and stored at 4° C. until used. The transformation of Agrobacterium tumefaciens was performed according to freeze-thaw method as follows: to 500 μl of Agrobacterium tumefaciens cell to be transformed was added 1 μg of plasmid pOgERS1-GUS DNA prepared in example 3; after mixed homogeneously, it was treated each for 5 minutes successively on ice, liquid nitrogen and at 37° C.; the bacterial suspension was then mixed with 1 ml YEB medium, and then cultured by shaking at 240 rpm at 28° C. for 3˜4 hours. Thereafter, the suspension was applied over a medium containing suitable antibiotics (50 μg/ml of kanamycin, 50 μg/ml of ampicillin) and incubated at 28° C. for 2 days.

After the transformation, a single colony of Agrobacterium tumefaciens containing plasmid pOgERS1-GUS prepared in Example 3 was inoculated in 5 ml YEB medium (0.5% beef extract, 0.1% yeast extract, 0.5% peptone, 0.5% mannitol, 0.05% MgSO₄, pH 7.5) containing suitable antibiotics (50 μg/ml of kanamycin, 50μg/ml of ampicillin) and was incubated by shaking at 240 rpm at 28° C. for 2 days. Then, the resulting suspension was poured into 250 ml YEB medium containing suitable antibiotics (50 μg/ml of kanamycin, 50 μg/ml of ampicillin) and incubated again at 28° C. at 240 rpm for 24 hours, followed by centrifuging at 4° C. 6,000 rpm for 10 minutes. The supernatant was discarded, and the pellet was suspended again in 200 ml of infiltration medium (½ MS, 5% sucrose, 0.044 μM BA, and 0.01% Silewet L-77, pH 5.7). Transformation of Arabidopsis thialana (Columbia) was carried out by a process modified from one described by Clough, and Bent (1998) as follows: Arabidopsis thialana (Columbia) transformant was placed upside down in Agrobacterium tumefaciens liquor, and soaked therein for 20 seconds; Arabidopsis thialana (Columbia) was then removed and kept wet for 24 fours; after about 3-4 weeks, its seeds were harvested.

### 3. Sowing and Screening of Transformant

The seeds of Arabidopsis thialana (Columbia) thus collected were rinsed several times with sterile water, treated with 70% ethanol for 2 minutes, and then with sterile waster containing 1% Clorox and 0.1% Tween-20 for 20 minutes. Thereafter, it was rinsed 4-5 times with sterile water each for 5 minutes. Then, the seeds were sown on a germinating modified medium (½ MS, 1% sucrose, 0.7% agar, 50 μg/ml of kanamycin, 50 μg/ml of ampicillin) to perform an anti-antibiotic progeny segregation test. The result was shown in Table 2. As the second pair of cotyledons was germinated after about 7-10 days, transformant could be obtained. The homozygote transformant progeny thus screened could be used then for promoter activity assays at different growth and development stages.

TABLE 2
Segregation of antibiotic resistance in the T2 progeny of plasmid
pOgERS1-GUS transformed Arabidopsis thialana.

| No. | Number of | Number of non-resistant | |
|---|---|---|---|
| T2 progeny | resistant clones | clones | X² value (3:1) |
| 1 | 84 | 37 | 2.01 |
| 3 | 67 | 32 | 2.83 |
| 5 | 62 | 15 | 1.25 |
| 6 | 90 | 27 | 0.23 |

Note:
X² value ≦3.841 indicated that at 5% level of confidence, no significant difference exited between individuals analyzed by chi-square distribution test.

### 4. Histochemical Staining of GUS

Leaves, inflorescences and siliques were clipped from the transformant and were soaked first in pre-treatment buffer solution [50 mM Na₃PO₄ (pH6.8), 1% Triton X-100] at 37° C. for 2 hours. Then, they were rinsed 2-3 times with buffer solution containing no Triton X-100 (50 mM Na₃PO₄, pH6.8), and were added with buffer (1 mM X-Gluc dissolved in 50 mM Na₃PO₄, pH6.8) containing X-Gluc (5-Bromo-4-chloro-3-indoxyl-beta-D-glucuronic acid). To this, a 25 inches-Hg vacuum was applied for 5 minutes, returned to atmospheric pressure for 5 minutes, and the procedure was repeated once again. Thereafter, it was allowed to react at 37° C. for 2 days. Finally, 70% ethanol was added to quench the enzymatic reaction and to discolor the tissue. The color presentation thereof was observed under microscope.

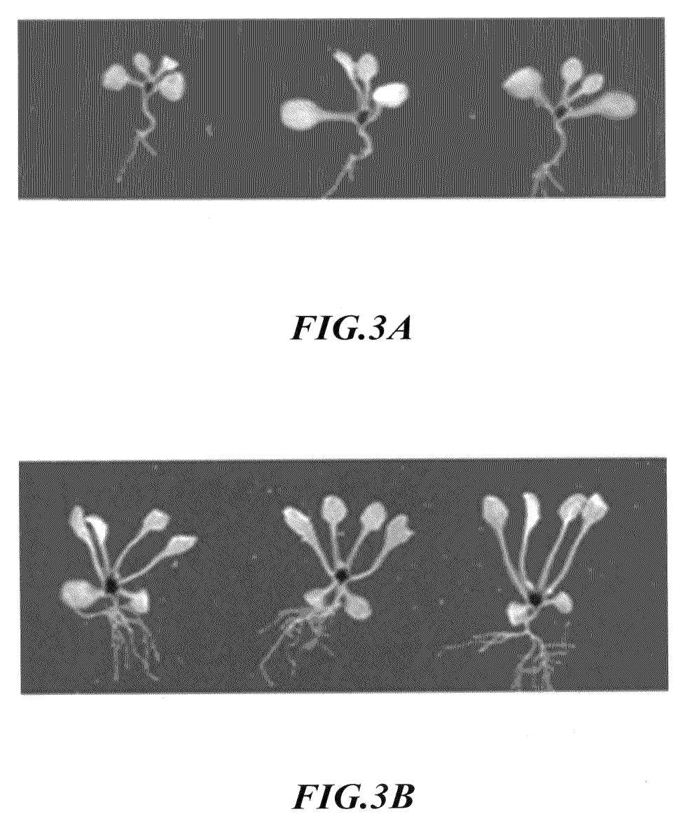

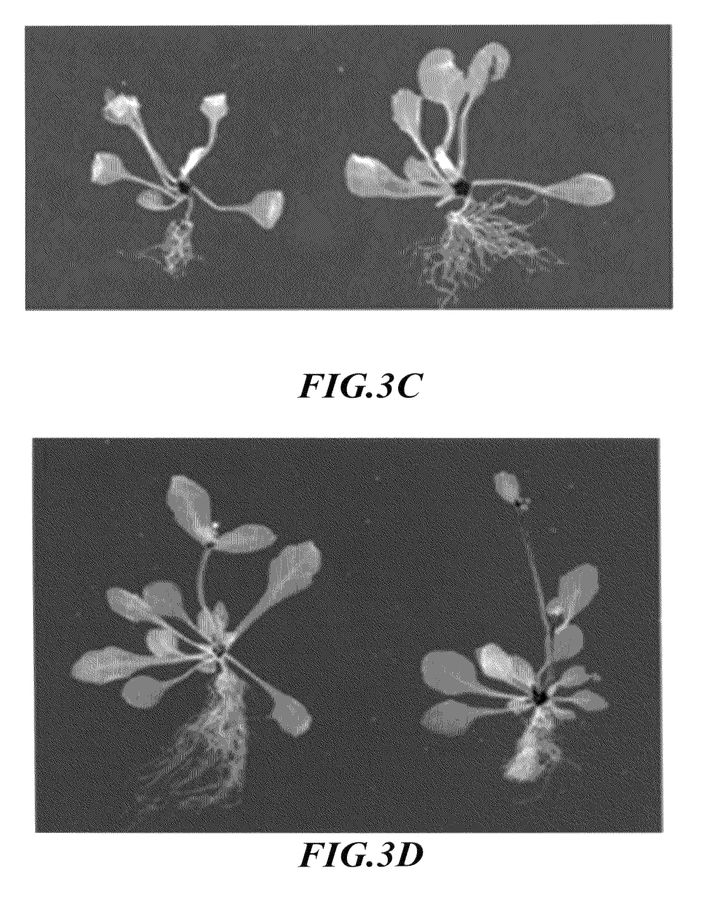

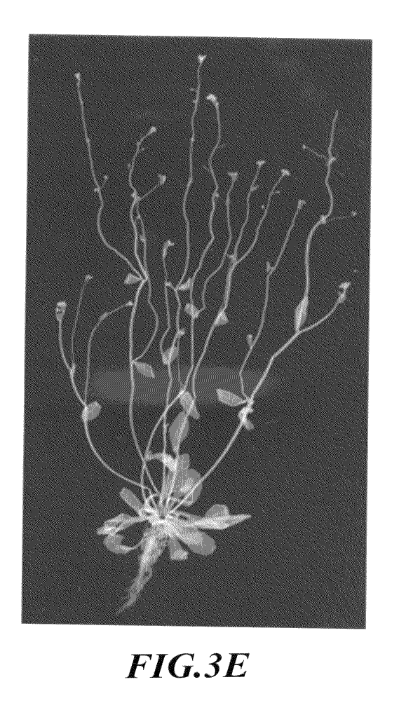

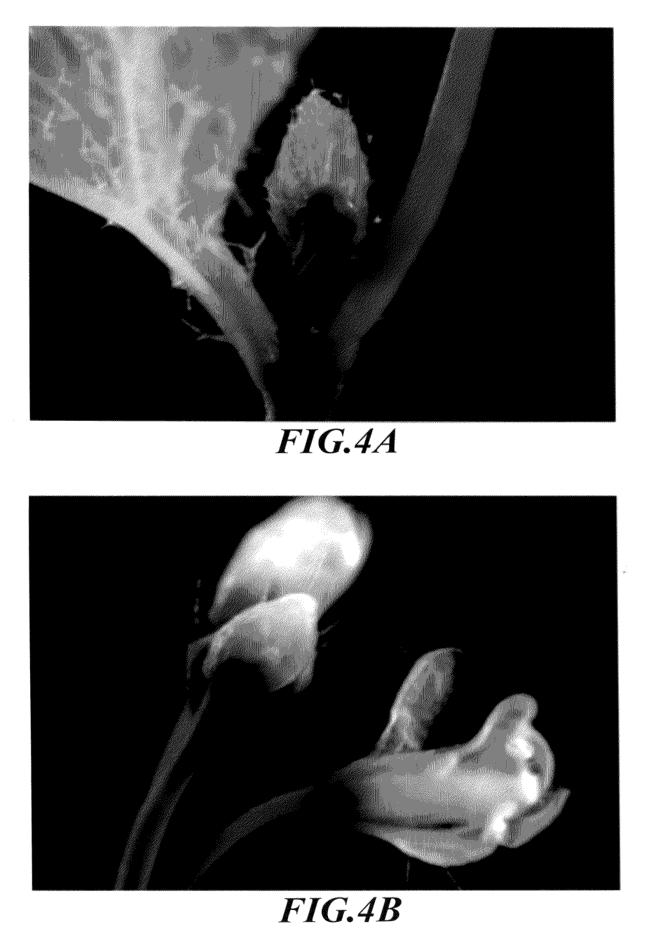

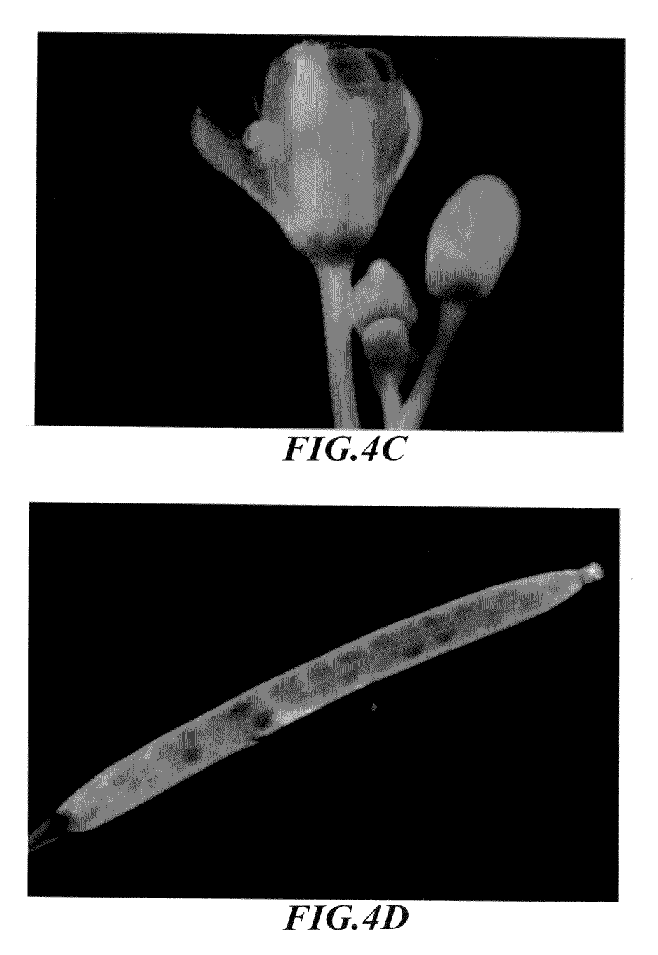

Analysis results of GUS activities from transformant progenies at different growth phases, namely, 10, 15, 20, 30 and 45 days after sowing, were shown in FIG. 3. As shown in FIG. 3A-3C, 10, 15, and 20 days after sowing, Arabidopsis thialana (Columbia) plantlets were in vegetative stage (leaf rosette stage). GUS concentrated its activity at and near the meristem region where the cell division takes place the most vigorously. As shown in FIG. 3D, at about 30 days after sowing, Arabidopsis thialana (Columbia) began to enter its reproductive stage and its GUS expression regions shifted progressively to buds at the top end as the inflorescence developed. On about 45 days after sowing, GUS activity would be observed at all inflorescence offshoots (as shown in FIG. 3E); buds and auxiliary bud (FIG. 4A); receptacles and part of pedicels (FIG. 4B); abscission zones involved in the aging and falling off of flowers (FIG. 4C); as well as abscission region involved in the falling off of siliques (FIG. 4D). These suggested that the promoter of said gene had significant activity in the tissue of young vigorous division as well as in tissue involved in the aging and falling off.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. A promoter capable of activating expression in plants, comprising the nucleotide sequence as shown in SEQ ID NO: 3.

2. A gene expression cassette comprising: the promoter as recited in claim 1; and a polynucleotide sequence having an open reading frame, wherein said polynucleotide sequence is operably linked to the Y terminal of said promoter, and, wherein said promoter can activate the transcription of said polynucleotide sequence in a plant having said gene expression cassette therein.

3. A gene expression vector, comprising the promoter of claim 1.

4. A plant, parts of organ or tissue, or cell of said plant that has been transformed to contain said gene expression cassette as recited in claim 2.


SEQUENCE LISTING (8 sequences)

SEQ ID NO: 1 - DNA, 2430 bp, Oncidium Gower Ramsey
  CDS (151)...(2046)<s300> Genebank/ AF2762332007-01-20
ctcatatcaaaccctagagcataccgctctcgtcggtccttcagaactttaggatttgtgccgaaattcattgaaaacgccatgttggtgtaccaggatatcctcaccaggatgtaggagaaagatagcaggtacagcagttctttagaaatggaaggctgtgattgcattgaaccacaatggcctgctgatgagcttttagtgaagtatcaatatatctctgatttcttcattgctcttgcctacttctccattccactggaactcatttatttcgtgaagaagtcttcatttttcccttatagatgggtgctcatacagtttggtgccttcattgtcctttgcggagcaacccacttgataaacttgtggacattcacaatgcactcgaggacagttgctatagtaatgactgtagcaaaagtttctactgctggtgtgtcatgtgcaacagccctgatgcttggtcacataattcctgatctggtaagcgttaaaacaagggagctttttcttaggaacaaagctgaagaacttgatagagagatgggtcttatacgcacacaggaagaaacagggaggcatgtgaggatgctcactcatgaaatacgaagtactcttgacagacacactatattgaagaccactcttgttgaacttggaaggactctagatttggcagaatgtgctttatggatgcctttacggactgggctccatctccagctttctcatactttaaacaacaaaatccctgttggatctgttgtttctattaacctccctatagtcaatcagatttttaatagcagtcgtgcaggtagaattccaaatacatgtccacttgcaaggtttcaaccttatacaggtagatatgtacctcaagaggttgttgcagtcagagtgccactcttacatttatcaaatttccaaataaatgattggcccgagctgtcacctaaaagttttgctgtgctggttttgatgctcccgtcagatagtgcaaggaaatggcatgcttatgaactagagcttgttgaggtagttgcagatcaggtcgcagttgctctttcacatgctgccatcttggaggaatccatgcgggcacgagatctactcatggatcaaaatgttgctttagatttagcacgaagggaagcagagatggccattcgtgcacgcaatgattttttagctgtcatgaaccatgagatgcggactcccatgcacgcaatcattgctctatcctcccttcttctcgaaactgaactgactcccgagcaacgtttgatggtcgaaaccatcttaaagagcagtaacttgctagcaacgctaatcaatgatgtcttagacctttcaaaactggaggatggcagcttcgagttggaggcaaccattttcaatcttcatactgtcttcagagaggctgtaaatttgataaaaccaatagcggctgtgaagaagttgtcattgtttgtgtctcttgctccggacttgccatcatgtgccattggagatgagaaacggcttctgcaaattatgcttaatgttgttgggaatgctgtaaagtttacaaaggagggtactatatttattactgtgaatattgcaaaacctgattcgttgagagacccagatttctaccctgcccctactgacgggcatttctatttaagagtacaggttaaggacaccggttgcggcataagtccacaggagttaccacacctcttcactaaatttgcgcatactcagaatggttctgacaaaggctactccggctctgggcttgggcttgccatttgcaagagattcgtcaatctcatgaaaggacacatttggcttgaaagtgaaggtattggcaaaggctgcaccaccattttcatagtgaagctgggcatgagtgaagatcccactcttcgctaccagcagaagttattgcccccaatccgagcaggccaagctgaagcagatccttttggtttaaagccggcggcactaaaggacgagcttatgccgaagattcggtatcagagaagtttgtaggttaaaattaagacgagttcttagaggcagttggatctgtgcttgtacactcgaaaataaagtcaatgtgaagtgcaggtagaagaaattttctctgatcccagaagcttccagatatcagcgcttgaactgaaaactgtaactgaaatattatcacaaattagctgtgggtttactctactgcttcaattccttgttaagcagttttactgaagagtgtatctaatctggaagagtggtgttcgatattaacttaagcttaggtgaagttatgttgtcactgtgaaatttatatggaaatcctttattagatgacacgctaaggagattgatctaggtgattaataaatcaaaatttaattagaaaaaaaaaaaaaaaaaaaa

SEQ ID NO: 2 - DNA, 2173 bp, Oncidium Gower Ramsey
ttggaacgcttccaaaaatccagcagctgccctctagcctcctcttttttgcagctaaaaggaatcattacctaatcttagattgggtcttttaattgcaggttattttaagtaaccggactgggtccagttaaagccggacggtgtccggttggacaccgcgcaatcagagcagttaccataacagaatcccgcgtcattttgaaatccggacgacgtccggttatgtccatacagagtccggtttagatgactagaccggacgctgtctggtcagaaatcaaccggacgattccgaataggtcggtgctgatgtatttttctcttcataggattaaccggacatcgtccggttatccataatcggaactgtccggttggcacagatgttgtatgttctccagcaacatgtcatccggaccgagtacgggtacctcaatcggaacggtccgggcgactcagacgtttcacttcttcaaagagacatccggaccgtgtccggatataaaaccggacatgtccggttgtgtcagatgattttctgcacagctcagtgaattttgtcaaatacaatttctcatttattaatatttaatgttatcaaaattagaggggctcatgagatcaacataatgcataaatgatactcgaatccctaattatttgaacaatttgctcaaattaacacaaccttgagtggtgcaactagtgcatttcaacctaattgtacatatgtttactaaaataaatacttttcttgcaattaatacatatatattgaaattttacgttcacaataaaactatttgctaattacatataataactaattttaaaaattttaattaaattgccacacttaaatttctccaaaaaaatattaataaaaataaaataaaatttgtataaatatatctaagcaaaacaatcatttagaacctaaactaataaatttgaatatattttgagagagataaaaaatgatgaaaattttccaaggccccgtttgtttcagcttttccaccacaatttttttttcatcattacacaaaaagttatttttcacctgtcattcacctgtcaccccctcacaacttttcaaccgtcattcaatgaatagtaaaaaatattccaccacaccacaccacacaaccgaaacaaacggggcccaaattaaaaacacaatatcttacaaattaatagatgaaatctaaataagtcagctttaaatggaagagtaatgagtacttttgattatatggataaaacaaaaaaaaatggagatttaagcaccaaacgatgtgccctatgtcacggtcgtgagtctcctatcatcactttctcaaatcaaacacataaaactaatttcttgtattcaattgcattcctcaaaacaaacacaacatatttcaaaatacaatgaatttattttacactgtattcttttttcacccaaccaaataacccctaaattaactccaatgtatttaattttatacaaattaatgaaaaataaattggataaattattttattgttcattcatttaagcataaattatagaaatttaaatttaattacaaataacttttagggtgcgtttgtttcggcgttgcgttgtgttgcggtggcactgtagctgcggtgacactgtagctgcggtggtactgtagctgaagtgtgttgcggtgtgaaaatttagttttggtgtttgaatatttgtactgtagctgcggtgtgttgtgatgtaagaaatgtgtagtttattattattattattattattattattattattattattattattattattattaatataataattattatgattattatttattattattattatcattattaattattaatttattattattattattattaattattattattattatttattattattattattattattattattatttttattatttaatattagtattaaaatataggagatgcagggtactgtagcaacgcaacacaacgcacctaaaaagcgggaattcaaacgtgtttgcgttgggttcaaagcagttttcaaacagggctttaataaaagaaattatttaaattattatagtgaactacattttggaagagtaatagccaagtctactgccgtctactttccctttccattttggtg

SEQ ID NO: 3 - DNA, 2323 bp, Oncidium Gower Ramsey
ttggaacgcttccaaaaatccagcagctgccctctagcctcctcttttttgcagctaaaaggaatcattacctaatcttagattgggtcttttaattgcaggttattttaagtaaccggactgggtccagttaaagccggacggtgtccggttggacaccgcgcaatcagagcagttaccataacagaatcccgcgtcattttgaaatccggacgacgtccggttatgtccatacagagtccggtttagatgactagaccggacgctgtctggtcagaaatcaaccggacgattccgaataggtcggtgctgatgtatttttctcttcataggattaaccggacatcgtccggttatccataatcggaactgtccggttggcacagatgttgtatgttctccagcaacatgtcatccggaccgagtacgggtacctcaatcggaacggtccgggcgactcagacgtttcacttcttcaaagagacatccggaccgtgtccggatataaaaccggacatgtccggttgtgtcagatgattttctgcacagctcagtgaattttgtcaaatacaatttctcatttattaatatttaatgttatcaaaattagaggggctcatgagatcaacataatgcataaatgatactcgaatccctaattatttgaacaatttgctcaaattaacacaaccttgagtggtgcaactagtgcatttcaacctaattgtacatatgtttactaaaataaatacttttcttgcaattaatacatatatattgaaattttacgttcacaataaaactatttgctaattacatataataactaattttaaaaattttaattaaattgccacacttaaatttctccaaaaaaatattaataaaaataaaataaaatttgtataaatatatctaagcaaaacaatcatttagaacctaaactaataaatttgaatatattttgagagagataaaaaatgatgaaaattttccaaggccccgtttgtttcagcttttccaccacaatttttttttcatcattacacaaaaagttatttttcacctgtcattcacctgtcaccccctcacaacttttcaaccgtcattcaatgaatagtaaaaaatattccaccacaccacaccacacaaccgaaacaaacggggcccaaattaaaaacacaatatcttacaaattaatagatgaaatctaaataagtcagctttaaatggaagagtaatgagtacttttgattatatggataaaacaaaaaaaaatggagatttaagcaccaaacgatgtgccctatgtcacggtcgtgagtctcctatcatcactttctcaaatcaaacacataaaactaatttcttgtattcaattgcattcctcaaaacaaacacaacatatttcaaaatacaatgaatttattttacactgtattcttttttcacccaaccaaataacccctaaattaactccaatgtatttaattttatacaaattaatgaaaaataaattggataaattattttattgttcattcatttaagcataaattatagaaatttaaatttaattacaaataacttttagggtgcgtttgtttcggcgttgcgttgtgttgcggtggcactgtagctgcggtgacactgtagctgcggtggtactgtagctgaagtgtgttgcggtgtgaaaatttagttttggtgtttgaatatttgtactgtagctgcggtgtgttgtgatgtaagaaatgtgtagtttattattattattattattattattattattattattattattattattattattaatataataattattatgattattatttattattattattatcattattaattattaatttattattattattattattaattattattattattatttattattattattattattattattattatttttattatttaatattagtattaaaatataggagatgcagggtactgtagcaacgcaacacaacgcacctaaaaagcgggaattcaaacgtgtttgcgttgggttcaaagcagttttcaaacagggctttaataaaagaaattatttaaattattatagtgaactacattttggaagagtaatagccaagtctactgccgtctactttccctttccattttggtgctcatatcaaaccctagagcataccgctctcgtcggtccttcagcactttaggatttgtgccgaaattcattgaaaacgccatgttggtgtaccaggatatcctcaccaggatgtaggagaaagatagcaggtacagcagttctttagaa

SEQ ID NO: 4 - DNA, 1872 bp, Oncidium Gower Ramsey
  CDS (1)...(1806)<s300> Genebank/ GI20885062000-12-01
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaa

SEQ ID NO: 5 - DNA, 29 bp, Artificial sequence
  Forward primer used in the synthesis of the 1st to 2283rd nucleotide sequence in SEQ ID No 3
tgcggatccttggaacgcttccaaaaatc

SEQ ID NO: 6 - DNA, 18 bp, Artificial sequence
  Reverse primer used in the synthesis of the 1st to 2283rd nucleotide sequence in SEQ ID No 3
ccaggatatcctcaccag

SEQ ID NO: 7 - DNA, 56 bp, Artificial sequence
  Forward primer used in the synthesis of the 2284th to 2323rd nucleotide sequence in SEQ ID No 3 and the full length sequence of SEQ ID No 4
gatgtaggagaaagatagcaggtacagcagttctttagaaatgttacgtcctgtag

SEQ ID NO: 8 - DNA, 27 bp, Artificial sequence
  Reverse primer used in the synthesis of 2284th to 2323rd nucleotide sequence in SEQ ID No 3 and the full length sequence of SEQ ID No 4
gcctcgggaattgctaccgagctcgaa